(19) United States
(12) Patent Application Publication
(10) Pub. No.: US 2015/0039016 A1
(43) Pub. Date: Feb. 5, 2015

(54) DEVICE AND METHOD FOR DEFLECTING EMBOLI IN AN AORTA

(71) Applicant: Keystone Heart Ltd., Caesarea (IL)

(72) Inventors: Gil Naor, Caesarea (IL); Yuval Shezifi, Haifa (IL)

(21) Appl. No.: 14/384,024

(22) PCT Filed: Mar. 7, 2013
(86) PCT No.: PCT/IL2013/000027

§ 371 Date: Sep. 9, 2014

Related U.S. Application Data
(60) Provisional application No. 61/608,855, filed on Mar. 9, 2012.

(51) Int. Cl. A61F 2/01

(52) U.S. Cl. CPC A61F 2/01; A61F 2002/016; A61F 2230/0006
USPC 606/200

(57) ABSTRACT

The invention features an intra-vascular device (10) which may include a filter (30), a filter insert (36), and a supporting structure (40) to hold a filtering element and may serve to filter or deflect emboli or other large objects from entering protected secondary vessels. The device may be capable of collapse along its longitudinal axis (80) for ease of delivery to the treatment site. The device may further be compatible with common delivery methods (e.g., TAVI procedures). Upon deployment, the device may be positioned in a middle area of a blood vessel (e.g., an aortic arch) near but not in contact with one or more second blood vessels (e.g., the branch arteries of the aorta). The supporting structure may be capable of pressing against the medial wall of a blood vessel (e.g., the aorta) and provide lift to the device so that a middle portion of the device is above a lateral plane of the device.

### CROSS-REFERENCE TO RELATED APPLICATION

This application claims benefit of U.S. Provisional Application No. 61/608,855 filed Mar. 9, 2012, which is hereby incorporated by reference in its entirety.

### FIELD OF THE INVENTION

The invention relates to devices for blocking emboli in an aorta from entering arteries.

### BACKGROUND OF THE INVENTION

Devices such as vascular filters or other devices may be inserted into a blood vessel prior to or during a procedure or at another time. Such devices may be inserted by way of a catheter that may be, for example, threaded through a vein or artery, and into for example an aorta or other vessel where the device may be released from the catheter and for example deployed. The device may filter, deflect, or block emboli or other objects from entering into a blood supply that feeds the brain.

### SUMMARY OF THE INVENTION

In one aspect, the invention features an intra-vascular device including a collapsed cylindrical portion including interspersed large and small diameter wires, having the collapsed cylindrical portion collapsed along its longitudinal axis to form a substantially flat filter including two layers; where the spaces between the small and large diameter wires are large enough to allow blood to pass and small enough to prevent large particles from passing; the filter is capable of insertion into the aorta and sized to simultaneously cover the left subclavian, left common carotid, or brachiocephalic arteries; and the large diameter wires provide structural support for the device.

In this aspect, the collapsed cylindrical portion can include a first end and a second end, each of the ends ending below a lateral plane of the lateral structure. The first end can include a hook configured from a wire of the collapsed cylindrical portion, the hook having a latch to hold a lasso brought into contact with the hook.

In any of the devices of the invention, the small diameter wires can be between 10-50 (e.g., 10, 20, 30, 40, or 50) microns in diameter and the large diameter wires can be between 80-200 (80, 120, 160, or 200) microns in diameter.

Also, any of the devices of the invention can include one or more (e.g., 1, 2, 3, 4, 5, or 6) wires that pass from a point distal to the collapsed cylindrical portion to a point proximal to the collapsed cylindrical portion, having the length of the wire extend downwards and/or upwards from the horizontal plane of the collapsed cylindrical portion.

In any of the devices of the invention, the wires can be connected (e.g., by crimping) at the distal and proximal ends to an internal tube. The internal tube can be capable of allowing a guidewire to pass through. In certain embodiments, the collapsed cylindrical portion can be connected to a delivery cable.

In other aspects, the device can also include an outer tube, having the capability of keeping the device in a compressed state until deployment.

In yet other aspects, the device can also include an internal filter material (e.g., braided, weaved, or clustered material) inside the collapsed cylindrical portion. The internal material may include Nitinol mesh.

In any of the devices of the invention, the collapsed cylindrical portion and/or filter can include Nitinol wire and/or Drawn Filled Tubing. The Drawn Filled Tubing can include an outer layer of Nitinol and/or a core including tantalum and/or platinum.

In another aspect, the lower or upper wire can include Drawn Filled Tubing. The Drawn Filled Tubing can include an outer layer of Nitinol and/or a core including tantalum and/or platinum.

In any of the devices of the invention, the device can further include a radiopacity marker (e.g., a bead or a clamp).

In another aspect, the invention features an intra-vascular device including a center region and two end regions, having: two end regions that are substantially cylindrical; a center region that is substantially flat; where the center region and two end regions can include wire braided in a continuous pattern, having the spaces formed by the braided wire define pores such that the pores in the two end regions are larger than the pores in the center region and the pores in the center region are large enough to allow blood to pass and small enough to prevent large particles from passing; and the device is capable of insertion into the aorta and sized to simultaneously cover the left subclavian, left common carotid, or brachiocephalic arteries.

In yet another aspect, the invention features an intra-vascular device including a cylindrical portion having interspersed wires, where: the edge of the cylindrical portion is folded over to form a cylindrical portion including at least two layers; the edge is closed; the spaces formed by the interspersed wires are large enough to allow blood to pass and small enough to prevent large particles from passing; and the device is capable of insertion into the aorta and sized to simultaneously cover the left subclavian, left common carotid, or brachiocephalic arteries.

In another aspect, the invention features methods of preventing passage of a particle from the aorta into the left sublclavian, left common carotid, or brachiocephalic arteries by inserting into an aorta any of the above-described devices such that the device prevents a particle from passing to the left subclavian, left common carotid, and brachiocephalic arteries. One or more wires can contact a medial surface of the ascending or descending aorta. The device can deflect and/or capture the particle, thereby preventing the particle from passing through the aorta into the left sublclavian, left common carotid, or brachiocephalic arteries.

As used herein, the term “collapsed cylindrical portion” refers to a region of the device that, when in isolation, has a circular or oval cross section and, when included in the devices of the invention, is collapsed along the longitudinal axis to form a two layer portion that is substantially flat in the perpendicular plane.

As used herein, the term “substantially flat” refers to a radius of curvature of no more than 80 mm (e.g., 10 mm, 20 mm, 30 mm, 40 mm, 50 mm, 60 mm, or 70 mm).

As used herein, the term “blood” refers to all or any of the following: red cells (erythrocytes), white cells (leukocytes), platelets (thrombocytes), and plasma.

As used herein, the term “large particles” refers to particles greater than 50 microns (e.g., 50, 150, 250, 350, 450, 550, 650, 750, 850, 950, or more microns) in the longest dimension. As used herein, the term “wires” refers any elongated structure (e.g., cords, fibers, yarns, filaments, cables, and threads) fabricated from any non-degradable material (e.g., polycarbonate, polytetrafluorothylene (PTFE), expanded polytetrafluorothylene (ePTFE), polyvinylidene fluoride, (PVDF), polypropylene, porous urethane, Nitinol, fluropolymers (Teflon®), cobalt chromium alloys (CoCr), and para-aramid (Kevlar®)), or textile (e.g., nylon, polyester (Dacron®), or silk).

As used herein, the term “delivery cable” refers to any delivery system used in interventional cardiology to introduce foreign bodies to a treatment site (e.g., catheters, guidewires, and wires).

As used herein, the term “provide structural support” refers to the property contributing to shape and stiffness of the device.

### BRIEF DESCRIPTION OF THE DRAWINGS

FIG. 1: is a schematic diagram of braided tube around a longitudinal axis (80) in accordance with an embodiment of the invention.

FIG. 2: is a schematic diagram of device (10) with collapsed cylindrical portion (19) and both ends (21 and 22) connected around a longitudinal axis (80), in accordance with an embodiment of the invention.

FIG. 3: is a schematic diagram of the collapsed cylindrical portion (19) of FIG. 2 compressed between two plates and shaped to form a substantial elliptical shape in one plane and substantially flat in the perpendicular plane, with one axis substantially longer that the one perpendicular to it (30) and one end is connected to the delivery cable (70), in accordance with an embodiment of the invention.

FIGS. 4A-4C are photographs showing a variety of mechanisms for connecting the intra-vascular device to a catheter.

FIG. 5 is a schematic diagram of a side view of a plunger for use in introducing intra-vascular devices of the invention into a subject, e.g., through a catheter.

FIG. 6: is a schematic diagram of a side view of the embodiment of FIG. 3 showing a possible filtering element (36) that is inserted in the filter (30) before it is compressed; this insert is used for filtering blood and can have an effective porosity between 50-950 microns (e.g., 50, 150, 250, 350, 450, 550, 650, 750, 850, 950, or more microns), in accordance with an embodiment of the invention.

FIG. 7A is a schematic diagram showing filter meshes of the indicated pore sizes.

FIG. 7B is a schematic diagram showing perforated films with the indicated patterns, sizes, and densities of pores.

FIG. 7C is a schematic diagram showing a filter mesh with a combination of DFT (Drawn Filled Tubing) and Nitinol wires.

FIG. 8A: is a schematic diagram of a top view of a possible structure (40) made of wires (41) (e.g., Nitinol) that may support the filter (30) and to force it against the branch opening for improved filtering; all wires can be relatively the same length to allow the structure to fold into a sheath, in accordance with an embodiment of the invention.

FIG. 8B: is a schematic diagram of a side view of the embodiment (40) showing the two wires (42) that support the filter (30) and the two wires (43) that are made to press the filter (30) against the artery wall, in accordance with an embodiment of the invention.

FIG. 9: is a schematic diagram of a top view of the embodiment (50) with the filter (30) assembled over the wire structure (40), in accordance with an embodiment of the invention.

FIG. 10A is a photograph of a cross section of DFT wire.

FIG. 10B is a schematic diagram of a filter mesh containing DFT wire.

FIG. 10C is a photograph of a radiopacity bead and clamp element for use in an embodiment of the invention.

FIG. 11: is a schematic diagram of a side view of the embodiment (50) placed in a vessel (60) having 3 branches, and embodiment (50) is in contact with both the medial surface of the aortic arch (71) and the outer arterial wall (72), in accordance with an embodiment of the invention.

FIG. 12: is a cross sectional view of FIG. 7 showing that the wires (41) of the supporting structure (40) follow the vessel wall (60) thus not interrupting blood flow or the passage of other therapeutic tools.

FIG. 13: is a schematic diagram of a side view of the embodiment (1) made of a braided wires in a cylindrical shape that is configured to have a center section with a substantially lower porosity (3) than the rest of the cylinder (2). In this portion (3), the wires are concentrated on one side of the braided tube and follow a curve along the surface of the tube, thus forming two eyelets that allow free passage through the embodiment (1) along its axis.

FIG. 14: is a schematic diagram of a top view of embodiment (1) showing the lower porosity section as well as the two eyelets (4) that form an opening through which a catheter may pass.

FIG. 15: is a schematic diagram of a side view of the embodiment (1) in a vessel with multiple branches (5). The section with the lower porosity is position against the branches thus filtering the blood that enters these branches.

FIG. 16: is a schematic diagram of embodiment (6) showing a braided cylinder that is folded over itself at one end, creating a smooth edge. Both ends are held together by the delivery cable, over a tubular member that facilitates the passage of a standard size guide wire. An additional filtering member can be placed between both levels of the braided cylinder.

FIG. 17: is a schematic diagram of embodiment (7) showing a braided cylinder of multiple wires, and configured to have between two to five sections (e.g., 1, 2, 3, 4, or 5 sections) of different porosities along its length, such as higher porosity on both sides (2) and a section of lower porosity in the center (3).

### DETAILED DESCRIPTION OF THE PREFERRED EMBODIMENTS

In general, the invention features an intra-vascular device for filtering or deflecting emboli or other large objects from entering a protected secondary vessel or vessels. The device of the invention may include a filter, a filter insert, and a supporting structure to hold a filtering element, and may serve to filter or deflect emboli or other large objects from entering protected secondary vessels. The device may be capable of collapse along its longitudinal axis for ease of delivery to the treatment site. The device may further be compatible with common delivery methods used in interventional cardiology (e.g., TAVI procedures). Upon deployment, the device may be positioned in a middle area of a blood vessel (e.g., an aortic arch) near but not in contact with one or more secondary blood vessels (e.g., the branch arteries of aorta). In another embodiment, the device may be positioned to contact the orifice of one or more secondary blood vessels. The supporting structure may be capable of pressing against the medial wall of a blood vessel (e.g., the aorta) and provide lift to the device so that a middle portion of the device is above a lateral plane of the device.

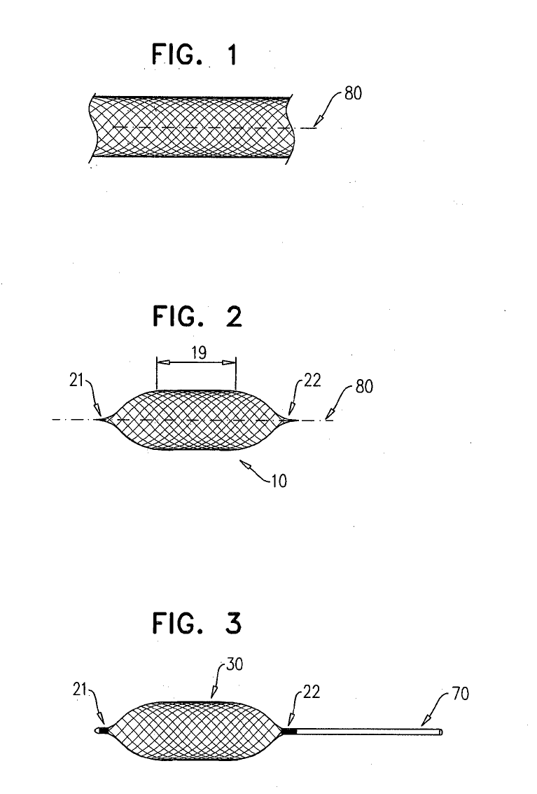

Reference is made to FIGS. 1 and 2: FIG. 1 is a schematic diagram of the braided tube, and FIG. 2 is a schematic diagram of a collapsed cylindrical portion (19) of device (10) with connected ends. Imaginary line (80) represents a theoretical lateral plane of device (10). In some embodiments, a lateral plane of device (10) may include an approximately horizontal line tracing a middle section of filtering collapsed cylindrical portion (19) along device (10) before the curves of end (21) and end (22). In some embodiments, device (10) may collapse along its longitudinal axis and form a substantially circular, elliptical, or elongated configuration of two layers. This bi-layer configuration may filter, deflect, or block emboli.

In embodiments where a wire mesh is used, the wire mesh can contain circular, elliptical, square, rectangular, or rhomboid shaped pores. Each dimension of the mesh pores can be, e.g., between 50 and 1000 microns (e.g., 70, 80, 90, 100, 200, 300, 400, 500, 600, or more microns). The wire mesh may comprise both small diameter wires between (e.g., 5, 10, 20, 30, 40, 50, 60, 70, 80, 90, or 100 microns in diameter) and large diameter wires (e.g., 60, 70, 80, 90, 100, 110, 120, 130, 140, 150, 160, 170, 180, 190, and 200 microns in diameter). The wires may be braided, weaved, clustered, knitted, or knotted. In certain embodiments, the stiffness of the intra-vascular device will be determined by the thickness of the large diameter wires. For example, the device can be stiffened by the inclusion of heavier gauge wire. Furthermore, multiple wires of a certain gauge can be wound together to increase the stiffness of the intra-vascular device (e.g., the collapsed cylindrical portion can include 2, 3, 4, 5, or more wires of to increase the stiffness of the intra-vascular device).

Reference is made to FIG. 3, a schematic diagram of the device (10) collapsed to form a substantially elliptical shape in one plane and substantially flat in the perpendicular plane. In some embodiments one or both of the intra-vascular device ends (21) and (22) are connected to (e.g., crimped, glued, soldered, clipped, latched, hooked, screwed or bonded) the delivery cable (70) (e.g., a catheter).

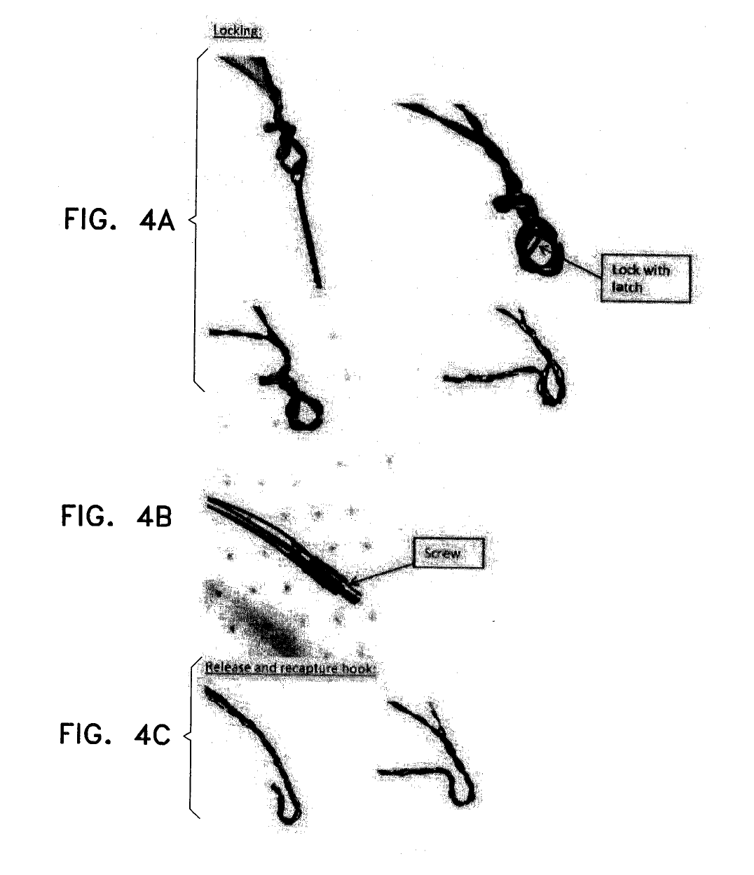

Reference is made to FIGS. 4A-4C. As described above, a variety of configurations can be used to connect the intra-vascular filter to a plunger (e.g., a plunger disposed within a catheter). FIG. 4A depicts a locking mechanism with a latch. FIG. 4B depicts a screw whereby the intra-vascular device can be mated with a screw on a plunger. FIG. 4C depicts a release and recapture hook for connecting the intra-vascular device with a plunger. In some embodiments, a hook may include a latch or wire strand that may be part of a wire strand that makes up the supporting structure, and that is in contact with a rest of the hook.

In other embodiments, a wire or catheter that may end in, for example, a loop, and may be threaded through latch so that the loop passes between a contact point of bend and curve. When so threaded, a wire or catheter fitted with a looped end may be clicked into a hook, and may securely push the device into place or pull the device out of position from a blood vessel (e.g., the aorta).

In some embodiments, the hook may end in a ball-tip so that strands from the collapsed cylindrical portion or supporting structure do not fray or scratch the vessel wall or the inner tube of a catheter.

In other embodiments, a clasp at an end of the device may be pressed into or onto a clasp at, for example, an end of a catheter and the two clasps may be joined by such pressing. In some embodiments, the device may be rotated clockwise or counter-clockwise respectively.

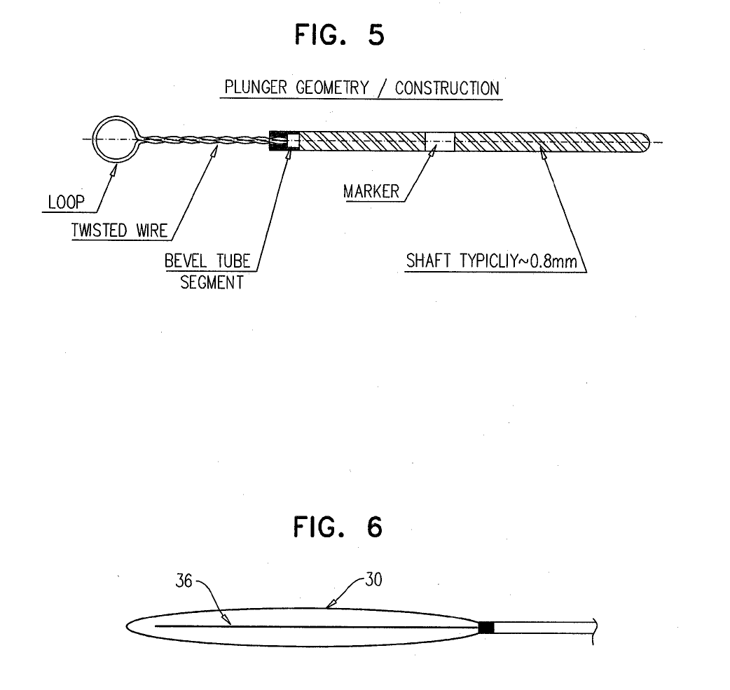

Reference is made to FIG. 5. The shaft or plunger for use in connection with the device can, e.g., terminate in a loop (as depicted in FIG. 5) or, e.g., a screw. In embodiments where a loop is present, the loop can be generated by winding two wires together leaving a loop at the distal end (FIG. 5). The shaft or plunger can, e.g., include a radiopaque element. Furthermore, the shaft or plunger can feature a rectilinear (e.g., square) or curved (e.g., oval or circular) cross section. Differences in cross sectional shape can have advantageous properties with respect to controlling the positioning of the intra-vascular device within the aorta.

In some embodiments, the distal end of the structure is attached to an internal tube that allows a standard size guide wire to pass through. In other embodiments, the proximal end is also attached to an internal tube that will allow a guide wire to pass through, and which is connected to the delivery cable (70).

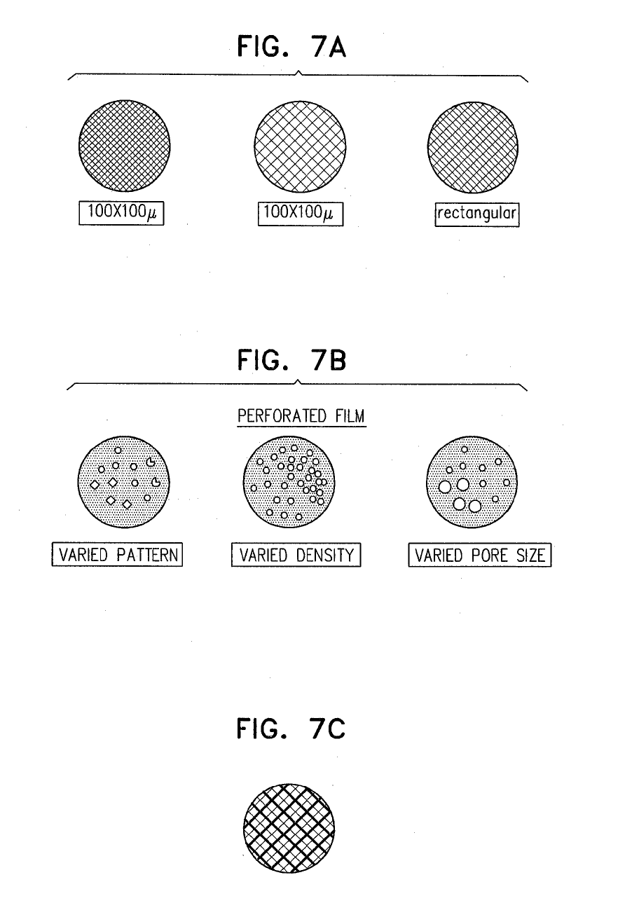

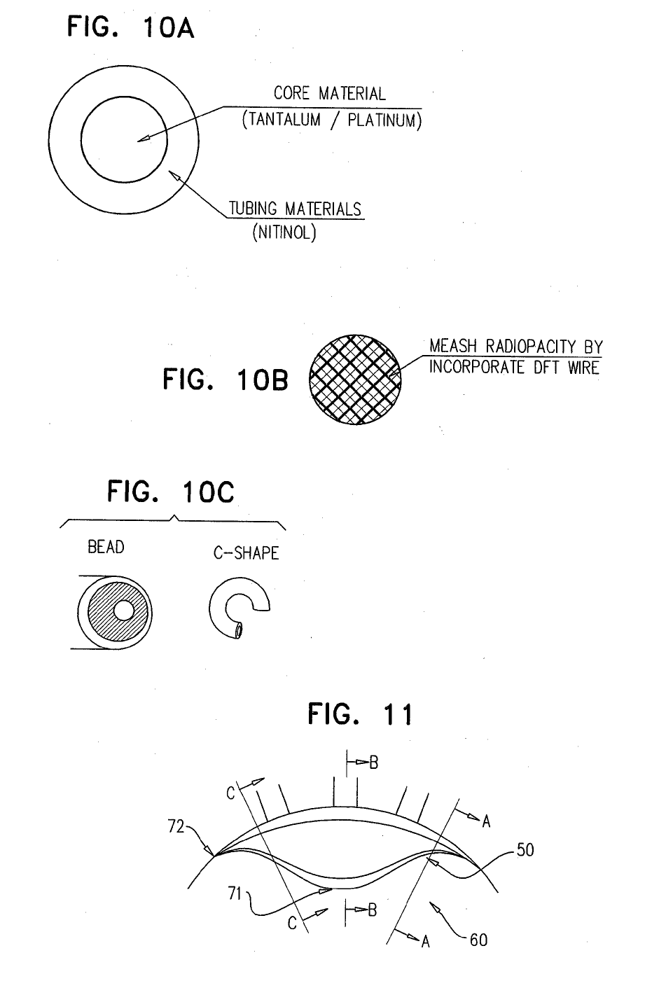

Reference is made to FIG. 6, a side view of an intra-vascular device including an additional filter element. In some embodiments, filtering insert (36) may be inserted into device (10). In other embodiments, the filtering insert (36) may be connected to device (10) (e.g., crimped, glued, soldered, clipped, latched, hooked, or bonded). In some embodiments, the filter insert (36) may be or include a fine wire netting or mesh (e.g., as depicted in FIGS. 7A and 7B), or perforated film (e.g., as depicted in FIG. 7B), such as a mesh or sheet having holes or porosity of 50-950 microns (e.g., 50, 150, 250, 350, 450, 550, 650, 750, 850, 950, or more microns). In embodiments where a perforated film is present, the pores can have constant or varied pore patterns, constant or varied pore densities, and/or constant or varied pore sizes (FIG. 7B). The filtering insert may be braided, weaved, clustered, knitted, or knotted. The filtering insert may be a non-degradable material (e.g., polycarbonate, polytetrafluorothylene (PTFE), expanded polytetrafluorothylene (ePTFE), polyvinylidene fluoride, (PVDF), polypropylene, porous urethane, Nitinol, fluropolymers (Teflon®), and para-aramid (Kevlar®)), or textile (e.g., nylon, polyester (Dacron®), or silk). The filtering insert may be a combination of material (e.g., the combination of DFT and Nitinol wires as depicted in FIGS. 10A and 10B). The filtering insert may also be coated with an anti-thrombogenic agent to prevent a thrombogenic reaction.

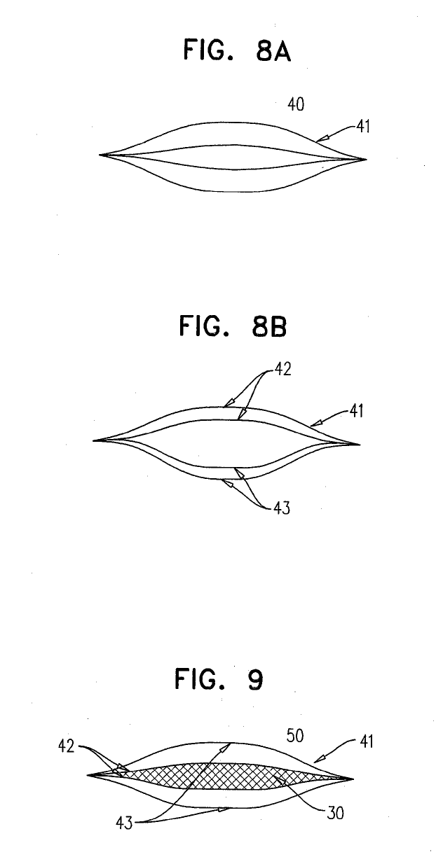

Reference is made to FIG. 8A, a schematic diagram of a supporting structure (40) present in certain embodiments, and FIG. 8B a side view of the supporting structure (40). In some embodiments, the supporting structure (40) is made of wires (41). The wires may be of relatively the same length and selected from a material such as Nitinol or other superelastic or shape memory alloy or material. Other materials may be used (e.g., DFT, Nitinol, tantalum, or platinum). In some embodiments, the two wires (42) support the filter (30) and the two wires (43) press the filter (30) against the outer arterial wall (72).

Reference is made to FIG. 9, a schematic drawing of a top view the embodiment (50), wherein the supporting structure (40) supports filter (30). Upper wires (42) provide structural support to the filter (30). The upper wires (42) may also exert a continuous force to keep the filter (30) substantially flat. Lower wires (43) may exert a continuous lift force on device (10) to keep filter (30) in pressure contact with the outer arterial wall (72).

In some embodiments, one or more of the wires that make up the filter (30) may be wound or braided around supporting wires (42), and no soldered or glued connections between the filter and supporting wires may be needed. In other embodiments, the filter may be attached to the supporting structure by adhesive or solder.

In some embodiments, it is desirable to incorporate radiopaque elements into the intra-vascular device. Such radiopaque elements can affix to, or incorporate into the skeleton of the intra-vascular device (e.g., affixed to device ends (21 or 22), a lower member, filter (30), filter material (36), or supporting wires (42 or 43)). The radiopaque element can be a bead or clamp (e.g., as depicted in FIG. 10C). In the case of a clamp, the element can be crimped onto the intra-vascular device. In any of the embodiments of the invention, radiopaque material can be incorporated into wire forming the supporting structure (40), filter (30), or filter insert (36) of the intra-vascular device (see, e.g., FIG. 10B). For example, portions of the skeleton or filter mesh can be constructed out of DFT wire. Such wire can contain, e.g., a core of tantalum and/or platinum and an outer material of, e.g., Nitinol (see, e.g., FIG. 10A).

In some embodiments, one or more wires (42 or 43) or filters (30 or 36) may include a lumen, such as, for example a hollow wire, which may hold for example a medicament that may be released into an artery or area where the device is implanted.

Reference is made to FIG. 11, a schematic drawing of a side view of the embodiment (50) placed in a vessel (60) (e.g., the aortic arch). In some embodiments device 10 may remain positioned in the blood vessel (e.g., aorta) while a procedure (e.g., transcatheter aortic valve implantation) is undertaken in, for example, a heart, blood vessel, or other in-vivo area, where such procedure entails tracing a lead such as a catheter through the blood vessel (e.g., aorta). In some embodiments, device (10) may be inserted or deployed through, for example, one of the branch arteries or directly through an artery in the area of the heart rather than by way of a catheter from a remote vessel. Upon deployment, installation, or release the upper wires (42) may meet the outer arterial wall (72), while the wires extending below the horizontal plane of the device (43) may contact the medial surface of the aortic arch (71).

In some embodiments, the device (10) and supporting structure (40) may be contracted when the device is folded in an outer tube, and the total area may expand when the filter is unfolded and deployed. Forward movement of outer tube will collapse the device, while retrograde movement will allow deployment. The length of the device may be from approximately 80 mm to 90 mm, or otherwise as may be necessary to approximate a distance between an upper wall of an ascending aorta, upstream of an opening of an innominate artery, and at an upper wall of a descending aorta downstream of an opening of a left subclavian artery. In some embodiments, the length of the device may be reduced to the length necessary to approximate a distance between upper wall of a descending aorta or an ascending aorta and the opening of the targeted artery (e.g., the left subclavian, left common carotid, or brachiocephalic arteries). The width of the device may be from 10 mm to 35 mm, or otherwise as may approximate an internal diameter of an aorta or the diameter of the take-off branches. The device may be inserted into the aorta or introduced into a blood vessel in a collapsed form, and may assume an extended form upon its release from a tube or other insertion or positioning mechanism.

In some embodiments, device (10) may assume a substantially elliptical or elongated shape. Other shapes may be used. Because the aortic anatomy can vary between individuals, embodiments of the intra-vascular device of the invention are shaped to adapt to a variety of aortic anatomies. The size of the device (10) and supporting structure (40) may be pre-sized and pre-formed to accommodate various patient groups (e.g., children and adults) or particular aortic anatomy. The device may vary in length from 10 mm to 120 mm (e.g., 25 mm, 45 mm, 60 mm, 75 mm, 90 mm, or 105 mm) and width from 5 mm to 70 mm (e.g., 10 mm, 20 mm, 30 mm, 40 mm, 50 mm, or 60 mm)

In an installed position, the intra-vascular device may be inserted into a first blood vessel. In some embodiment, such first blood vessel may be or include an aorta, though the device may be inserted into other vessels. The filter (30) of the device may be positioned so that an opening of a second blood vessel is covered by the filter, so that for example large particles are filtered, blocked, or deflected from entering, for example, the left subclavian, left common carotid or brachiocephalic arteries, or any combination thereof (e.g., the left subclavian, left common carotid and brachiocephalic arteries; the left subclavian and left common carotid arteries; left common carotid and brachiocephalic arteries; and the left common carotid and brachiocephalic arteries). The space under filter (30) may allow unfiltered blood to pass by the branch arteries of the aorta. Such space in the aorta that is left below the filter means that not all blood passing through the aorta is subject to the filtering or deflecting process of filter (30). In an installed position, the device remains substantially flat (e.g., does not exceed a radius of curvature of 80 mm (e.g., 10 mm, 20 mm, 30 mm, 40 mm, 50 mm, 60 mm, or 70 mm).

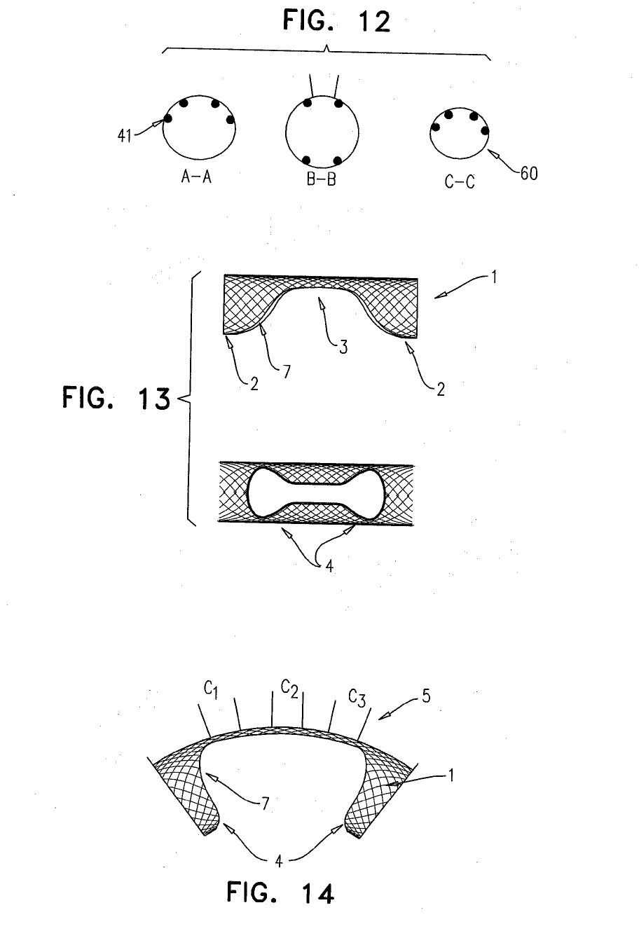

Reference is made to FIG. 12, a diagram of the contact points between the supporting structure (40) and filter (30) and the vessel wall. Points at which the implanted device may contact the outer arterial wall and the medial surface of the aortic arch are represented at three locations within the vessel. In some embodiments, vessel blood flow (e.g., arterial blood flow) is not interrupted. In some embodiments, therapeutic tools may pass through the aortic arch unimpeded.

Reference is made to FIG. 13, a diagram of the braided tube, open at either end, with diverting wire (7). The interspersed wires of the braided tube are diverted along path (7) to form two ellipsoidal openings within the braided tube. The diameter of the ellipsoidal openings do not fully sever the braided tube, but instead create three distinct portions. The ends (2) of the braided tube retain the normal braided configuration, while the wires of braided portion (3) are forced to occupy a smaller area thereby creating an area with a porosity between 100 and 500 microns (e.g., 100, 200, 300, 400, or 500 microns).

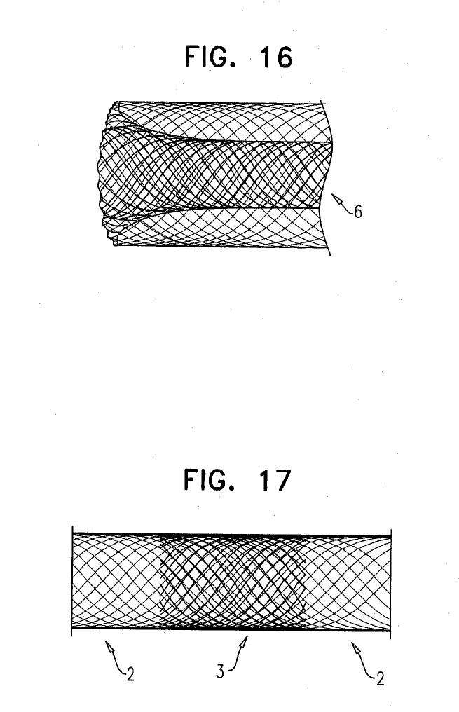

Reference is made to FIGS. 14 and 15: FIG. 14 is a diagram of a braided tube with two ellipsoidal openings (4), and FIG. 15 is a diagram of the device (1) within the aortic arch. The ellipsoidal openings (4) are created by the diversion of the braided material along path (7). Compressed portion (3) is pressed against multiple branch orifices (5). The openings (4) create an unobstructed channel below portion (3) within the vessel (e.g., aortic arch) to allow for unimpeded passage of materials (e.g., blood or surgical tools).

Reference is made to FIG. 16, a diagram of a braided tube where the end of the tube is folded over the braided tube thereby creating a braided tube of multiple layers (e.g., 1, 2, 3, 4, or 5). This multi-layered braided tube may serve as a filter. In other embodiments, a filter insert may be placed between the two layers. The filtering insert may be braided, weaved, clustered, knitted, or knotted. The filtering insert may be a non-degradable material (e.g., polycarbonate, polytetrafluorothylene (PTFE), expanded polytetrafluorothylene (ePTFE), polyvinylidene fluoride, (PVDF), polypropylene, porous urethane, Nitinol, fluropolymers (Teflon®), and para-aramid (Kevlar®)), or textile (e.g., nylon, polyester (Dacron®), or silk). The filtering insert may be a combination of material (e.g., the combination of DFT and Nitinol wires as depicted in FIGS. 10A and 10B). The filtering insert may also be coated with an anti-thrombogenic agent to prevent a thrombogenic reaction. The end of the braided tube (2) may be connected to a delivery cable to allow for deployment and retraction from the outer tube.

Reference is made to FIG. 17, a schematic diagram of the distinct filtering regions of the braided tube. Portions (2) posses a specific porosity (e.g., 50, 100, 200, 300, 400, 500, 600, 700, 800, 900, or 1000 microns) while center portion (3) posses an independent porosity (e.g., 100, 200, 300, 400, 500, 600, or 700 microns) The device may have between two to five portions of distinct porosities (e.g., 1, 2, 3, 4, or 5). The order of the porous portions may vary without limitation (e.g., the lowest porosity section may be at the proximal end, the distal end, or the center). In some embodiments, the distinct porosity portions is achieved by grouping at least two (e.g., 2, 3, 4, 5, 6, 7, or 8) wires on a single bobbin during an initial step in the braiding process, and later separating them onto distinct bobbins.

In still other embodiments, device (10) may be adapted for use with other embolism protection devices (e.g., those described U.S. application Ser. Nos. 13/300,936 and 13/205,255, in U.S. Publication Nos. 2008-0255603 and 2011-0106137, and in U.S. Pat. Nos. 8,062,324 and 7,232,453), each of which is hereby incorporated by reference in its entirety.

All publications and patents cited in this specification are incorporated herein by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An intra-vascular device comprising;
a collapsed cylindrical portion comprising interspersed large and small diameter wires,
wherein:
(i) said collapsed cylindrical portion is collapsed along its longitudinal axis to form a substantially flat filter comprising two layers;
(ii) the spaces between said small and large diameter wires are large enough to allow blood to pass and small enough to prevent large particles from passing;
(iii) said filter is capable of insertion into the aorta and sized to simultaneously cover the left subclavian, left common carotid, or brachiocephalic arteries; and
(iv) said large diameter wires provide structural support for said device.

2. The device as in claim 1, wherein the collapsed cylindrical portion comprises a first end and a second end, each of said ends ending below a lateral plane of said lateral structure.

3. The device as in claim 1, wherein said first end includes a hook configured from a wire of said collapsed cylindrical portion, said hook having a latch to hold a lasso brought into contact with said hook.

4. The device as in any of claims 1 to 3, wherein said small diameter wires are between 10-50 microns in diameter and said large diameter wires are between 80-200 microns in diameter.

5. The device as in any of claims 1 to 4, additionally comprising one wire that passes from a point distal to the collapsed cylindrical portion to a point proximal to said collapsed cylindrical portion, wherein the length of said wire extends downwards from the horizontal plane of said collapsed cylindrical portion.

6. The device as in any of claims 1 to 4, additionally comprising at least two wires that pass from a point distal to the collapsed cylindrical portion to a point proximal to said collapsed cylindrical portion extending downwards from the horizontal plane of said collapsed cylindrical portion.

7. The device as in any of claims 1 to 4, additionally comprising one wire that passes from a point distal to the collapsed cylindrical portion to a point proximal to said collapsed cylindrical portion, wherein the length of said wire extends upwards from the horizontal plane of said collapsed cylindrical portion.

8. The device as in any of claims 1 to 4, additionally comprising at least two wires that pass from a point distal to the collapsed cylindrical portion to a point proximal to said collapsed cylindrical portion extending upwards from the horizontal plane of said collapsed cylindrical portion.

9. The device as in any of claims 1 to 8, wherein said wires are connected at the distal and proximal ends to an internal tube.

10. The device as in claim 9, wherein said wires are connected by crimping to the internal tube.

11. The device as in claim 9, wherein said internal tube is capable of allowing a guidewire to pass through.

12. The device as in claim 9, wherein said collapsed cylindrical portion is connected to a delivery cable.

13. The device as in any one of claims 1 to 12, wherein said device additionally comprises an outer tube, wherein said outer tube is capable of keeping said device in a compressed state until deployment.

14. The device as in any of claims 1 to 13, wherein said device additionally comprises an internal filter material inside said collapsed cylindrical portion.

15. The device as in claim 14, wherein the internal filter material comprises braided, weaved, or clustered material.

16. The device as in any of claims 1 to 15, wherein said collapsed cylindrical portion comprises Nitinol wire.

17. The device as in any of claims 1 to 16, wherein said internal filter material comprises Nitinol mesh.

18. The device as in any of claims 1 to 17, wherein said device further comprises Drawn Filled Tubing.

19. The device as in claim 18, wherein said Drawn Filled Tubing comprises an outer layer of Nitinol.

20. The device as in claim 18 or 19, wherein said Drawn Filled Tubing comprises a core comprising tantalum and/or platinum.

21. The device as in any of claims 1 to 20, wherein said filter comprises Drawn Filled Tubing.

22. The device as in claim 21, wherein said filter Drawn Filled Tubing comprises an outer layer of Nitinol.

23. The device as in claim 21 or 22, wherein said filter Drawn Filled Tubing comprises a core comprising tantalum and/or platinum.

24. The device as in any of claims 1 to 23, wherein said lower wire or upper wire comprises Drawn Filled Tubing.

25. The device as in claim 24, wherein said lower wire or upper wire Drawn Filled Tubing comprises an outer layer of Nitinol.

26. The device as in claim 24 or 25, wherein said lower wire or upper wire Drawn Filled Tubing comprises a core comprising tantalum and/or platinum.

27. The device as in any of claims 1 to 26, wherein said device further comprises a radiopacity marker.

28. The device as in claim 27, wherein said radiopacity marker is a bead or a clamp.

29. A intra-vascular device comprising a center region and two end regions, wherein:
(i) said two end regions are substantially cylindrical;
(ii) said center region is substantially flat;
(iii) said center region and two end regions comprise wire braided in a continuous pattern, wherein the spaces formed by the braided wire define pores such that the pores in said two end regions are larger than the pores in said center region and the pores in said center region are large enough to allow blood to pass and small enough to prevent large particles from passing; and
(iv) said device is capable of insertion into the aorta and sized to simultaneously cover the left subclavian, left common carotid, or brachiocephalic arteries.

30. An intra-vascular device comprising;
a cylindrical portion comprising interspersed wires, wherein:
(i) the edge of said cylindrical portion is folded over to form a cylindrical portion comprising at least two layers;
(ii) said edge is closed;
(iii) the spaces formed by said interspersed wires are large enough to allow blood to pass and small enough to prevent large particles from passing; and
(iv) said device is capable of insertion into the aorta and sized to simultaneously cover the left subclavian, left common carotid, or brachiocephalic arteries.

31. A method of preventing passage of a particle from the aorta into the left sublclavian, left common carotid, or brachiocephalic arteries comprising deploying the device of any of claims 1-30 in said aorta such that:
said device prevents a particle from passing to the left subclavian, left common carotid,
and brachiocephalic arteries.

32. The method of claim 31 such that:
(i) said one or more wires contact a medial surface of the ascending or descending aorta.

33. The method of claim 31, wherein said device deflects and/or captures said particle, thereby preventing said particle from passing through the aorta into the left sublclavian, left common carotid, or brachiocephalic arteries.
